(19) United States
(12) Patent Application Publication
(10) Pub. No.: US 2015/0080661 A1
(43) Pub. Date: Mar. 19, 2015

(54) WATER BOTTLE ADAPTER FOR COUPLING AN ENDOSCOPE TO A WATER BOTTLE

(71) Applicant: UNITED STATES ENDOSCOPY GROUP, INC., Mentor, OH (US)

(72) Inventors: CHRISTOPHER J. KAYE, Concord, OH (US); ALISON STREIFF, Painesville, OH (US); GARY E. MANN, Mentor, OH (US); JOSEPH MRVA, Kirtland, OH (US)

(73) Assignee: UNITED STATES ENDOSCOPY GROUP, INC., Mentor, OH (US)

(21) Appl. No.: 14/456,783

(22) Filed: Aug. 11, 2014

Related U.S. Application Data
(62) Division of application No. 13/464,263, filed on May 4, 2012, now abandoned.
Application No. 13/464,263 is a continuation-in-part of application No. 13/093,989, filed on Apr. 26, 2011.
(60) Provisional application No. 61/327,997, filed on Apr. 26, 2010.

(51) Int. Cl. A61B 1/00; A61B 1/12

(52) U.S. Cl. CPC A61B 1/00128; A61B 1/12
USPC 600/158

(57) ABSTRACT

An adapter for coupling a water bottle to an endoscope including a cap manufactured from a thermoplastic elastomer material. The cap is removably secured to an associated water bottle. The cap includes a first port and a second port. The adapter further includes a scope connector manufactured from the thermoplastic elastomer and/or themoset elastomer having a first scope connector port and second scope connector port, wherein the scope connector is configured to be frictionally coupled to an endoscope. A first supply tube couples to the first port of the cap and the first scope connector port and a second supply tube coupled to the second port of the cap and the second scope connector port.

### RELATED APPLICATIONS

This application is a continuation-in-part of U.S. patent application Ser. No. 13/093,989 filed Apr. 26, 2011, which claims the benefit of U.S. Provisional Application No. 61/327,997 filed Apr. 26, 2010, both of which are hereby incorporated herein by reference in their entirety.

### TECHNICAL FIELD

The present disclosure relates to endoscope systems. More particularly, the present disclosure relates to an adapter for a water bottle that may be disposable, wherein the adapter is operative for coupling the water bottle to an endoscope system in order to deliver sterilized water to the endoscope instrument.

### BACKGROUND

Endoscopic instruments have been developed to provide surgeons with an internal view of the organ or body passage requiring treatment. Such endoscopes typically have channels through which a miniaturized forceps or other device, commonly called flexible instruments, are inserted and advanced. The endoscope assembly includes an elongated flexible cable equipped at one end with an eyepiece or other viewing mechanism and at the other end with an optical head. Only the head is directly and externally connected to the instrument. The cable transmits images or image-producing signals from the illuminated operative site to the viewing mechanism so that the surgeon will have visual confirmation of the action of the instrument's working end.

The cable also provides a flow passage for the delivery of fluid (liquid or gas) for irrigation or other purposes. In conventional practice, it is necessary to provide the optic head with a flow of sterile water. The passage of the sterile water across the optic head prevents the buildup of materials on the optic head.

A conventional endoscope includes a plurality of connectors that can suitably receive various fittings. For example, the connector can include a connector orifice that receives an air inlet and a water inlet. As such, the air and water are delivered through the connector to optic head of the endoscope.

Unfortunately, there is usually great expense associated with the delivery of such sterile water to the endoscope. In past practice, the sterile water has been provided from a water bottle that is directly connected to a tube. The tube generally will have a fitting at one end so as to allow the tube to be connected to the air/water inlet of the endoscope connector. Typically, the fitting will include an inner tube and an outer tube. The outer tube extends into the water bottle. The outer tube is connected to the cap of the water bottle. In normal practice, air is delivered through the area between the inner tube and the outer tube so as to pressurize the interior of the water container. This will force water to flow through the tube and into the endoscope at a desired rate.

After usage, the water bottle, the tubing, and the associated fittings are sterilized. This creates a considerable wasteful expense to the hospital. If the water bottle is sterilized, there is a considerable labor expense associated with the autoclaving of the bottle. There is also the possibility of residual contaminants residing in the area of connection between the tubes and the bottle.

### SUMMARY OF INVENTION

Conventional adapters for coupling a water bottle to the endoscopic assembly are made of hard, non-pliable materials that rely on multiple structural components to maintain a suitable seal. As a result, manufacturers are required to take elaborate steps in the manufacturing process to ensure a suitable seal between the air and fluid passage from the water bottle to the endoscope assembly. These additional steps include, for example, requiring ultrasonic welding of small components together, designing complex connecting structures, which are usually implemented with a gasket (or O-ring) to ensure a suitable seal, and the like. With conventional adapters, these additional steps were required to ensure an adequate seal. However, the additional steps are extremely costly and provide no additional value to the customer.

One aspect of the disclosure relates to an adapter for coupling a water bottle to an endoscope including: a cap manufactured from a thermoplastic elastomer and/or an thermoset elastomer, wherein the cap includes an interior surface of the cap, which may include threads or may be formed without threads, for removably securing the cap to an associated water bottle and the cap includes at least a first port and a second port; a scope connector manufactured from the thermoplastic elastomer and/or the thermoset elastomer having a first scope connector port and second scope connector port, wherein the scope connector is configured to be frictionally coupled to an endoscope; a first supply tube coupled to the first port of the cap and the first scope connector port; and a second supply tube coupled to the second port of the cap and the second scope connector port.

Another aspect of the disclosure relating to an adapter for coupling a water bottle to an endoscope including: a cap manufactured from a thermoplastic elastomer and/or a thermoset elastomer, wherein the cap includes an interior surface of the cap for removably securing the cap to an associated water bottle based on a material interface between the interior surface of the cap and the water bottle, and the cap includes a first port and a second port; a scope connector manufactured from the thermoplastic elastomer and/or a thermoset elastomer having a first scope connector port and second scope connector port, wherein the scope connector is configured to be frictionally coupled to an endoscope; a first supply tube coupled to the first port of the cap and the first scope connector port; and a second supply tube coupled to the second port of the cap and the second scope connector port.

To the accomplishment of the foregoing and related ends, the disclosure, then, comprises the features hereinafter fully described and particularly pointed out in the claims. The following description and the annexed drawings set forth in detail certain illustrative embodiments of the disclosure. These embodiments are indicative, however, of but a few of the various ways in which the principles of the disclosure may be employed.

### BRIEF DESCRIPTION OF THE DRAWINGS

FIG. 1 is an environmental view of an exemplary endoscopic irrigation system.

FIG. 2 is a side perspective view of an exemplary adapter.

FIG. 3 is a cross-sectional view of an exemplary cap.

FIG. 4 is a cross-sectional view of another exemplary cap.

FIG. 5 is a side perspective view of an exemplary scope connector.

FIG. 6 is a cross-sectional view of the exemplary scope connector.

FIG. 7 is a cross-sectional view of yet another exemplary cap.

FIG. 8A is a top-view of another exemplary cap.

FIG. 8B is a perspective-view of the exemplary cap of FIG. 8A.

FIG. 9 is a cross-sectional view of still another exemplary cap.

FIG. 10A is a top-view of another exemplary cap.

FIG. 10B is a perspective-view of the exemplary cap of FIG. 10A.

### DETAILED DESCRIPTION

Embodiments will now be described with reference to the drawings, wherein like reference numerals are used to refer to like elements throughout. It will be understood that the figures are not necessarily to scale.

Aspects of the present disclosure relate to an adapter for coupling a water bottle to an endoscope. As discussed below, the adapter includes a cap for connecting the adapter to a water bottle and a scope connector for coupling the adapter to the endoscope. The adapter includes an air tube and a water tube coupled to air and water ports located on the water bottle cap and the scope connector, respectively. Optionally, the adapter may include more ports. For example, the adapter may include a CO2 port and/or a water tube for pump-style irrigation. The CO2 port would provide CO2 access through tubing and a one way valve with luer lock for connection to a CO2 source (such as a CO2 insufflator). The one way valve would allow CO2 to pressurize the bottle and force water to flow to the distal tip of the endoscope. In operation, air forced through the air tube from the endoscope into the water bottle causes water to flow from the water tube into the endoscope assembly field. The water bottle cap and/or the scope connector are made from a thermoplastic elastomer (TPE) and/or a thermoset elastomer that have sufficient pliability and/or are capable of frictionally engaging with other structures (e.g., endoscope port, air tube, water tube, etc.) in such a manner to provide a substantially air tight seal and at the same time allow a user to easily install and remove the adapter. One way of providing this pliability and frictional engaging capability is by over-molding or otherwise affixing a soft elastomer onto at least portions of a hard-plastic adapter body. Such a process provides a suitable seal without requiring ultrasonic welding of small components or the designing of complex coupling structures.

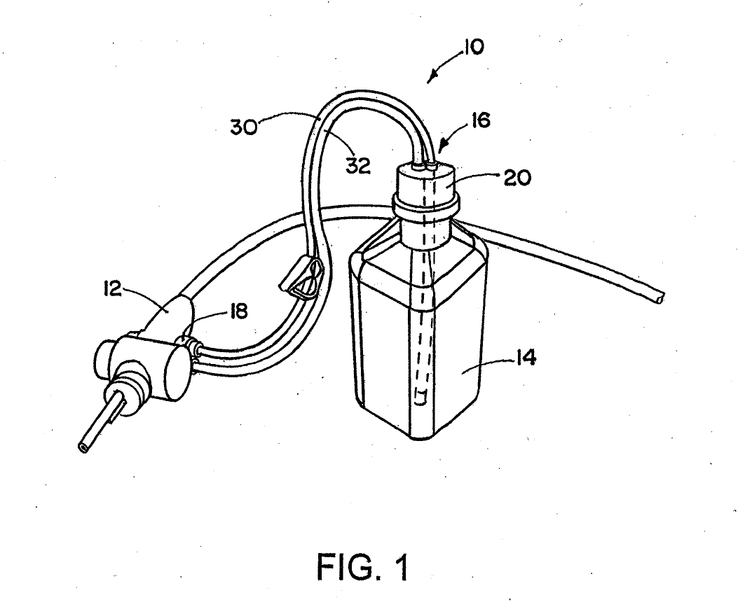

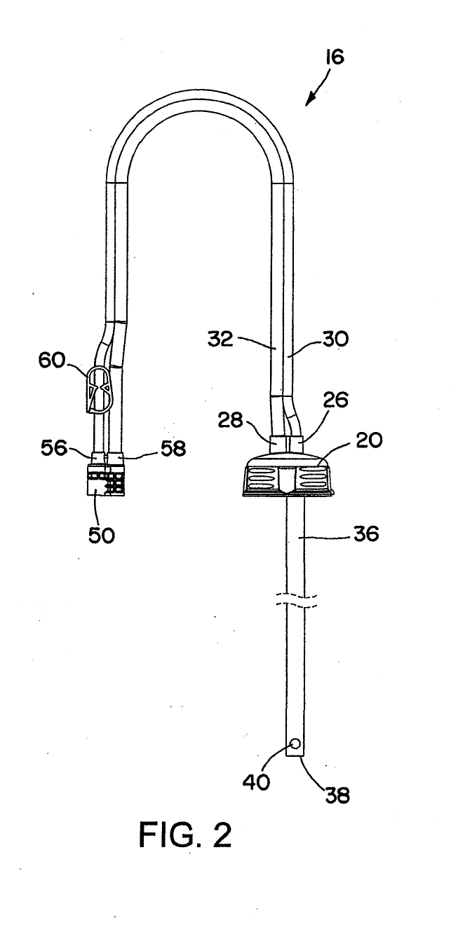

Referring to FIG. 1, an exemplary system 10 in accordance with aspects of the present disclosure is illustrated. The system 10 includes an endoscope 12 coupled to a water bottle 14 through an adapter, identified generally in FIG. 1 as reference numeral 16. The adapter 16 is illustrated in additional detail in FIG. 2.

The endoscope 12 may be any type of endoscope that is manufactured by any manufacturer. Preferably, the endoscope is operative to receive dual tubes (e.g., one for air and one for water) at an endoscope connector 18.

The water bottle 14 may be any size and/or type of water bottle. The water bottle 14 may be, for example, a one liter water bottle of a conventional type used in hospitals. The water bottle 14 is conventionally filled with sterile water. It is typical to use sterile water since the water will pass to the interior of the human body during the process of cleaning the optic head of the endoscopic instrument. The water bottle 14 generally has an externally threaded neck. In normal use, a cap is threadedly secured to the threaded neck, so as to prevent leakage or dispensing of the water from the interior of the bottle during transportation and storage of the water bottle.

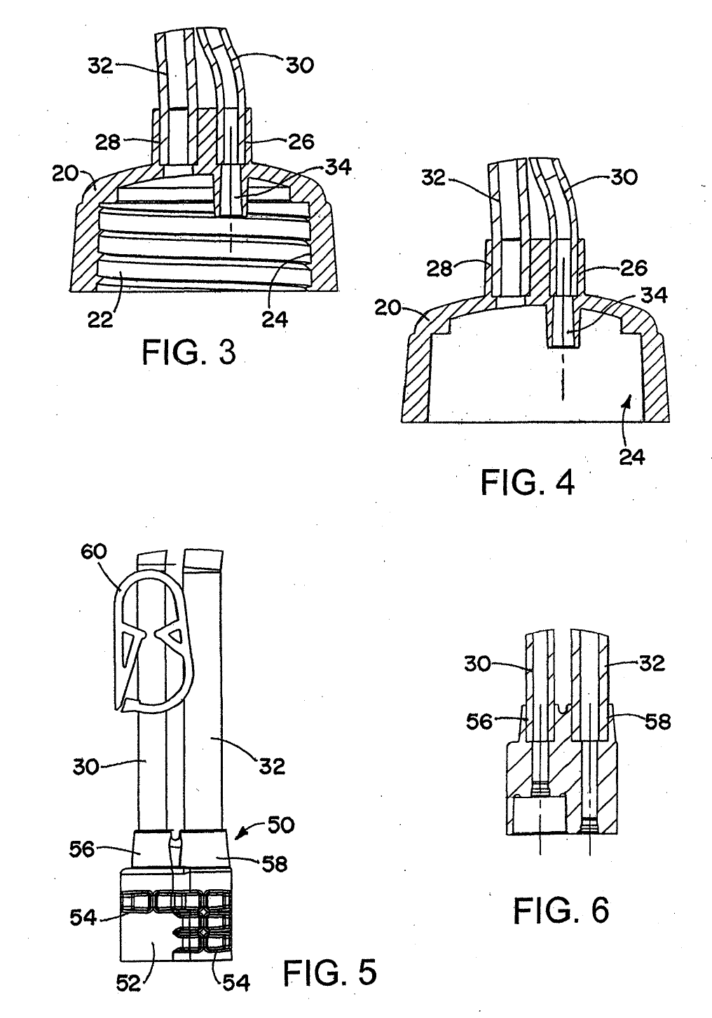

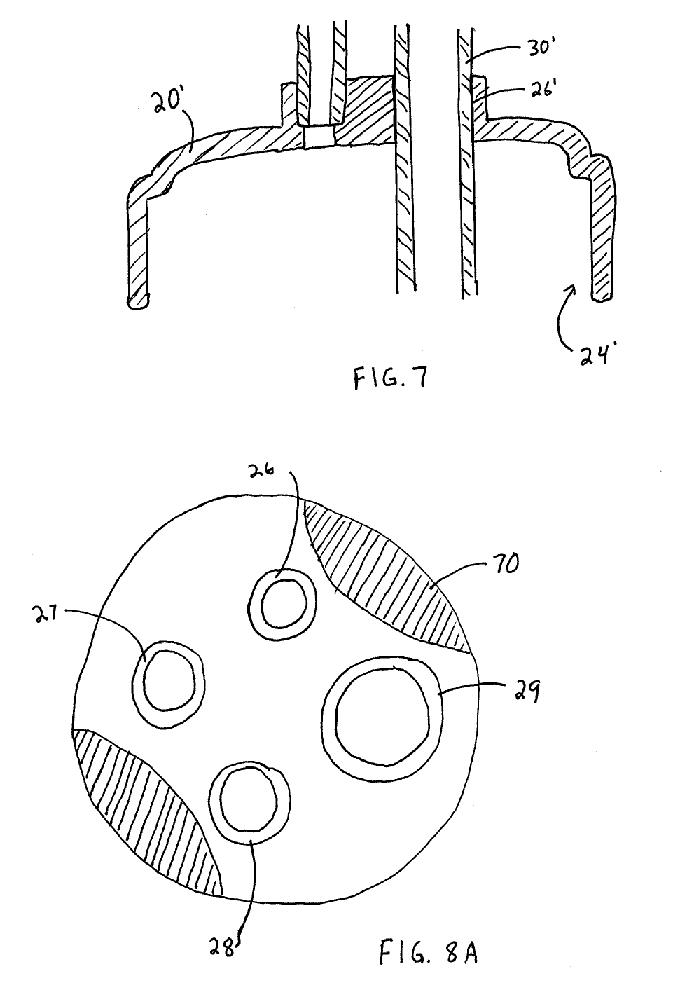

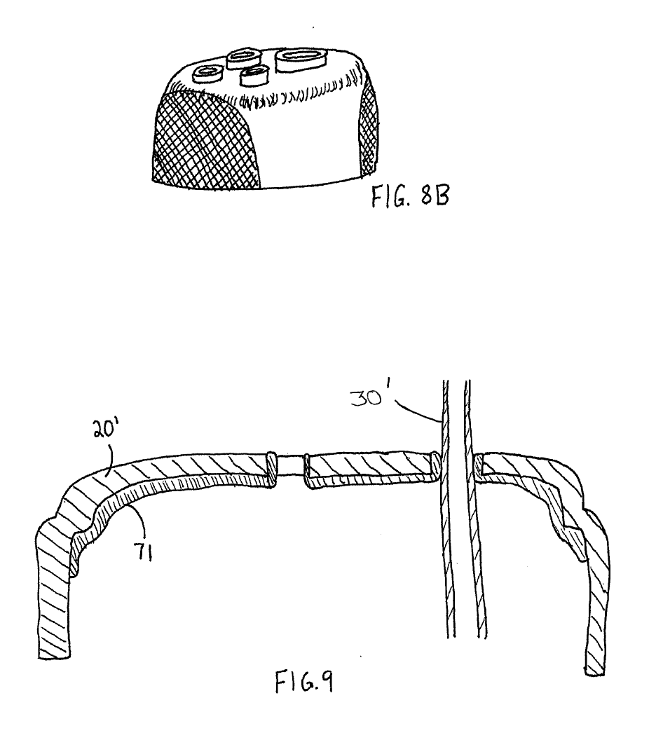

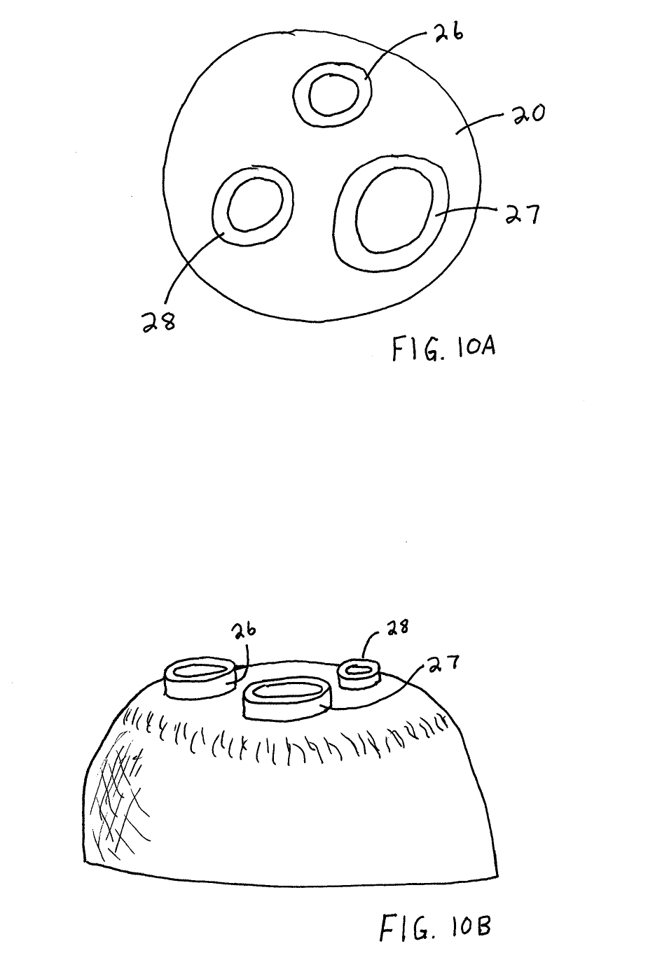

In order to affect the use of the adapter 16, it is necessary for the water bottle cap to be threadedly removed from the exterior of the neck of water bottle 14. Adapter 16 may then be secured to the water bottle 14. Referring to FIG. 2, the adapter 16 includes a cap 20. As discussed below, the cap 20 may be formed with one or more threads or without threads. In one embodiment, the cap 20 may include one or more threads 22 on an interior surface 24 of the cap for removably securing the cap to the water bottle 14, as shown in FIG. 3. The interior threads 22 of the cap may be sized to fit over various shapes and sizes of exterior threads that may exist on the neck of the water bottle 14 or the threads 22 may be designed especially to mate with one type of water bottle thread type.

In one embodiment, the threads 22 may be designed so as to match the variation in threads between the various brands of water bottle. In another embodiment, the threads 22 may be buttress threads having a four milliliter pitch (the distance between the threads). As such, even though the thread designs of the various brands of water bottles are different, the particular pitch and shape of the threads 22 are configured so as to allow the cap 20 to be attached to more than one type of water bottle.

In another embodiment, the cap 20 may be formed without threads, as illustrated in FIG. 4. The cap 20 includes an interior surface for removably securing the cap to the water bottle 14 (e.g., an outside diameter of the water bottle opening). The cap is secured to the bottle based on the ability of the elastomeric material to be compliant and conform to the outside bottle diameter in order to form a seal. For example, the cap 20 may stretched or otherwise temporarily enlarged to fit over the water bottle 14. When the stretching or enlarging force is released, the cap will frictionally engage with the water bottle.

In such embodiment, the cap 20 may take any desirable size and shape, so long as the interior surface of the cap is capable forming a suitable interface with the water bottle. In another embodiment, an over-molded soft elastomer seal 71 may be brought into tight engagement with the bottle opening by a snap-fit or by a thread fit of a hard cap body onto the mating surface of the bottle.

In contrast to many prior art water bottle caps that rely on gaskets or a series of complicated connection structures to establish an air and water tight seal with the water bottle, the cap 20 may be manufactured from a thermoplastic elastomer (TPE) and/or a thermoset elastomer that establishes an air water tight seal with the water bottle 14, without relying on any additional sealing mechanism. The cap 20 may be a fully soft cap or a combination of a hard cap body 20′ and a soft elastomer seal 71 affixed (for example, by over-molding) to the hard cap body. It is also contemplated that the cap may be formed from more than one piece. For example, a hard cap body may take the form of a threaded or snap-fit ring in which the hollow portion of the ring is filled with a hard and/or soft disc that may contain the ports of the cap.

Exemplary materials that may be used in accordance with aspects of the present disclosure include: styrenic block copolymers, polyolefin blends, elastomeric alloys (TPE-v or TPV), thermoplastic polyurethanes, thermoplastic copolyester and/or thermoplastic polyamides, silicone, natural and synthetic rubbers, and equivalents. Examples of products that come from block copolymers group are Styroflex (BASF), Kraton (Shell chemicals), Pellethane (Dow Chemical), Pebax, Arnitel (DSM), Hytrel (Du Pont) and more. While there are now many commercial products of elastomer alloy, these include: Dryflex ([VTC TPE Group]), Santoprene (Monsanto Company), Geolast (Monsanto), Sarlink (DSM), Forprene (So.F.Ter. S.p.a.), Alcryn (Du Pont) and Evoprene ([AlphaGary]). In one embodiment, the cap 20 may be made of flexible polyvinyl chloride (PVC), for example. One of ordinary skill in the art will appreciate that the above list is illustrative and not intended to limit the scope of the present disclosure.

The cap 20 includes a first port 26 and a second port 28 for introduction of water and air (or other gas) into the bottle 14 though the cap 20, as illustrated in FIGS. 2-4. The first port 26 and the second port 28 extend generally from an outer surface of the cap and are formed through the cap 20 in order to provide pathways for water and/or air to enter and/or exit the ports. In actual use, a controlled flow of air is maintained through the second port 28 so as to control the flow of water through the interior first port 26. If more water flow is desired, then greater air pressure is delivered through the second port 28 to the interior of the water bottle 14. If less water flow is desired, then less air pressure is applied. If no water flow is desired, then less air pressure is delivered and the bottle is merely pressurized to a point where quick flow can be generated on demand.

The first port 26 and the second port 28 can be formed integrally with the cap 20. A first supply tube 30 may be coupled to the first port 26. A second supply tube 32 is coupled to the second port 28. The supply tubes 30, 32 may be secured to the respective ports in any desirable manner. In one embodiment, the supply tubes may be secured to the respective port during formation of the cap 20 and ports 26, 28, for example, in an over-molding/dual shot manufacturing process. In such a process, the supply tubes 30, 32 may be placed in a mold that forms the cap 20, along with the first and second ports 26, 28. When the molding process is completed (e.g., using an injection molding process, etc.), the first supply tube 30 is secured to the first port 26 and the second supply tube 32 is secured to the second port 28 during formation of the cap with first and second ports.

In another embodiment, the supply tubes 30, 32 may be secured to the first and second ports 26, 28 after formation of the ports. In such case, an adhesive may be used to secure the first supply tube 30 to the first port 26 and the second supply tube 32 to the second port 28. An exemplary adhesive may be, for example, cyanocrylate (CA), which may be supplied by Loctite, Dymax, for example. In another embodiment, one or more of the supply tubes 30′ may pass through the respective port 26′ as is shown in FIG. 7. The tube 30′ may be sealed to the port 26′ through the inherent softness of the tube, may mate with an over-molded seal 71, as shown in FIG. 9, and/or may be secured to the port 26′ as described above.

The cap 20 may also include a third port 34, which is illustrated in FIGS. 3 and 4. The third port 34 can be fluidly connected to the first port 26, such that fluid may flow from third port 34 through the first port 26. As illustrated in FIG. 2, a water bottle supply tube 36 may be secured to the third port 34 by an adhesive and/or any other desirable mechanism. In use, the water bottle supply tube 36 may be inserted through the neck of the water bottle 14. As the cap 20 is threadedly secured to the water bottle 14, the water bottle supply tube 36 is lowered into the water bottle 14. The water bottle supply tube 36 may have an end 38 that rests at or near the bottom of the water bottle 14 in order to draw water from the water bottle 14. In one embodiment, the water bottle supply tube 36 has one or more voids 40 formed near the end 38 for facilitating the drawing of water from the water bottle 14 (14 (to ensure that fluid flow is not prevented by suction anchoring the tube to the container wall). In another embodiment, an anchor (not shown) may be affixed to the water bottle supply tube 36 so as to ensure the end 38 remains at or near the bottom of the water bottle 14.

Another embodiment of the present disclosure is directed to the first supply tube 30 terminating at or near the bottom of the water bottle 14. In such an embodiment, the third port 34 disclosed may not be needed, as such the third port 34 is optional.

The first and second supply tubes 30, 32 may have one end that is secured within the first and second ports 26, 28, as illustrated in FIGS. 3 and 4. In another embodiment, the ends of the first and second supply tubes 30, 32 may extend past the first and second ports 26, 28.

The cap 20 or 20′ may also include third and or fourth separate ports 27 and 29 for providing third and fourth access points to the bottle. Port 27 may, for example, be provided for a CO2 tube, and port 29 for another water tube. The second water tube may be, for example, a tube providing pump-style or drip-style irrigation. Another possible utilization for port 29 is the delivery of a procedure enhancing additive (foaming agent, cleansing material, medication, etc.).

Optionally, one or more of the ports (in one preferred embodiment, ports 27 and 29) may be closed by a cover. Optionally, the cover may be a removable cover. The cover may be molded in place, for example, and may be removable by, for example, providing perforation, scoring, or any other suitable means.

The cap may further include over-molded sections 70 to provide a more comfortable grip and/or more friction with a user's hand.

The adapter 16 further includes a scope connector 50, as illustrated in FIGS. 5 and 6. The scope connector 50 generally has an outer housing 52 that is configured to mate with endoscope connector 18. As shown in FIG. 5, the scope connector 50 is formed to be matingly inserted into the endoscope connector 18. In order to enhance the seal of the scope connector 50 within the endoscope connector 18, the scope connector 50 may include one or more ridges 54 formed in the housing 52. The one or more ridges 54 may be of varying size. The housing 52 and one or more ridges 54 are sized to provide an air and water tight seal with the endoscope connector 18. In particular, the housing 52 of the scope connector is configured and size to frictionally engage with the endoscope connector 18 in such a manner to form an air tight seal.

Like the cap 20 discussed above, the scope connector 50 may be manufactured from a thermoplastic elastomer (TPE) and/or thermoset material, such that an air and water tight seal may be formed between the endoscope connector 18 and the scope connector 50 without requiring any additional sealing mechanism (e.g., a gasket, O-ring, etc.). One of ordinary skill in the art will readily appreciate that the cap 20 and the scope connector 50 may be manufactured from the same or different materials.

The scope connector 50 includes a first scope connector port 56 and second scope connector port 58. The ports 56, 58 are coupled to first supply tube 30 and the second supply tube 32, respectively in such a way to establish two independent passageways between the water bottle 14 and the endoscope 12. For example, the first supply tube 30 is coupled to the first port 26 of the cap 20 and the first scope connector port 56. Likewise, the second supply tube 32 is coupled to the second port 28 of the cap 20 and the second scope connector port 58.

In operation, when water is desired at the endoscope operative sight, air may be forced through second scope connector port 58 though the second supply tube 32 through the second port 28 and into the water bottle 14. As air is forced into the water bottle 14, water is drawn through the water bottle supply tube end 38, through the third port 34 into to the first port 26 of the cap 20. From the first port 26, the water travels through the first supply tube 30 to the first scope connector port 56 for use by the endoscope 12.

The supply tubes 30, 32 may be secured to the respective ports 56, 58 in any desirable manner. In one embodiment, the 30, 32 may be secured to the respective port during formation of the port 56, 58, for example, in an over-molding manufacturing process. In such a process, the supply tubes 30, 32 may be placed in a mold that forms the scope connector 50, along with the first and second ports 56, 58. Such that when the mold is completed, the first supply tube 30 is secured to the first port 56 and the second supply tube 32 is secured to the second port 58 during formation of the cap with first and second ports.

In one embodiment, the supply tubes 30, 32 may be secured to the first and second ports 56, 58 through the use of an adhesive, as discussed above with respect to securing the supply tubes 30, 32 to the first and second ports 26, 28 of the cap 20.

Referring to FIG. 6, the first and second supply tubes 30, 32 may have one end that is secured within the first and second ports of the scope connector 56, 58 to facilitate fluidic communication between the endoscope 12 and the adapter 16 with the water bottle 14.

At least one of the supply tubes 30, 32 may include a pinch clamp 60, as illustrated in FIGS. 2 and 5. The pinch clamp 60 may be used by the operator to stop or start the fluid flow through the liquid supply tube (e.g., the first supply tube 30) at any time.

Although the disclosure has been shown and described with respect to a certain embodiment or embodiments, it is obvious that equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the disclosure. In addition, while a particular feature of the disclosure may have been described above with respect to only one or more of several illustrated embodiments, such feature may be combined with one or more other features of the other embodiments, as may be desired and advantageous for any given or particular application.

1.-20. (canceled)

21. An adapter for coupling a water bottle to an endoscope comprising:
a cap having a first port and a second port and formed by a hard body, and a soft seal over-molded to an inside surface of the cap, wherein the interior surface of the cap is adapted for removably securing to an associated water bottle;
wherein the soft seal includes a first protrusion extending into the first port and a second protrusion extending into the second port.

22. The adapter of claim 21, wherein the cap is manufactured from a thermoplastic elastomer and/or a thermoset elastomer.

23. The adapter of claim 21, wherein the soft seal is manufactured from a soft elastomer.

24. A cap for coupling a water bottle to an endoscope by tubing, the cap comprising:
a hard body manufactured from a first material, the hard body having an exterior surface defining a first port and a second port, and an interior surface adapted for removably securing to a water bottle; and
a seal manufactured from a second material, the seal being over-molded and affixed to the interior surface of the hard body and having a first protrusion extending into and the length of the first port and a second protrusion extending into and the length of the second port.

25. The cap of claim 24, wherein a tube inserted through the first port mates with the first protrusion.

26. The cap of claim 24, wherein a tube inserted through the second port mates with the second protrusion.

27. The cap of claim 24, wherein the soft seal includes a flange extending longitudinally in an opposite direction of the first port and the second port.

28. The cap of claim 24, wherein the first material is a thermoplastic elastomer and/or a thermoset elastomer.

29. The cap of claim 24, wherein the second material is a soft elastomer.

30. The cap of claim 24, wherein the cap further includes a plurality of soft grip portions over-molded to an exterior surface of the cap, wherein each of the plurality of soft grip portions is manufactured from the first material.
